(19) United States
(12) Patent Application Publication
(10) Pub. No.: US 2004/0241252 A1
(43) Pub. Date: Dec. 2, 2004

(54) Pharmaceutical compositions for oral administration comprising lithium carbonate, processes of making the same, and methods of administering the same

(76) Inventors: Christopher Charles Abney, Mt. Sterling, KY (US); Brandon Scott Hampton, Winchester, KY (US); James Russell Norton, Lexington, KY (US); Daniel Joseph Schave, Winchester, KY (US); George Ronald Tomaich, Lexington, KY (US)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/619,766

(22) Filed: Jul. 15, 2003

Related U.S. Application Data
(60) Provisional application No. 60/474,096, filed on May 29, 2003.

(51) Int. Cl. A61K 33/00

(52) U.S. Cl. 424/715

(57) ABSTRACT

The present invention pertains to a controlled release solid dose formulations of lithium carbonate, comprising lithium carbonate, optional pharmacologically acceptable excipients, lubricants including stearic acid, sodium stearyl fumerate, calcium stearate, and magnesium stearate, optionally glycine, and sodium carboxymethylcellulose. Tablet forms are compressed at various pressures. The sodium carboxymethylcellulose and optionally glycine increases the dissolution rate profiles for lithium carbonate formulations, particularly for those formulations stored for extended periods of time and at varying conditions of heat and humidity. A process of formulating such compositions, comprising the steps of mixing lithium carbonate with excipients, top spraying a solution of sodium carboxymethylcellulose and glycine onto the lithium mixture in a fluid bed granulator, milling, and pressing the resultant compound into tablets is also described. The formulations of the invention are useful in a method of treatment of Bipolar Disorder (Manic Depressive Disorder).

### CROSS-REFERENCE TO RELATED APPLICATIONS

The present application claims priority to Provisional Application No. 60/474,096 filed May 29, 2003, the disclosure of which is incorporated herein by reference in its entirety.

### TECHNICAL FIELD

The present invention relates to pharmaceutical formulations; in particular to solid dosage forms of pharmaceutical compositions comprising lithium carbonate, and methods of preparing such compounds.

### BACKGROUND OF THE INVENTION

Bipolar disorder is a chronic, cycling, and often debilitating mental illness that affects more than 2 million adults in the United States, or about 1 percent of the adult population 18 and over in any given year, according to the National Institute of Mental Health. Bipolar disorder typically develops in late adolescence or early adulthood, although first occurrence at younger and older ages has been recognized. Bipolar disorder is often not recognized as a medical illness, and affected persons may suffer for years before their condition is properly diagnosed and treated. Bipolar disorder causes sometimes dramatic mood swings, from overly “high” and/or irritable to sad and hopeless, and then back again, often with periods of normal moods in between. Signs and symptoms of mania, sometimes called a manic episode, include increased energy, activity, and restless; overly good, euphoric mood; extreme irritability; racing thoughts and fast, pressured speech; distractibility; difficulty sleeping and decreased need for sleep; and poor judgment that may manifest itself as spending sprees, abuse of drugs, intrusive or aggressive behavior, or other harmful or dangerous activities. Signs and symptoms of depression associated with bipolar disorder, sometimes called a depressive episode, include lasting sad, anxious, or empty mood; feelings of hopelessness, guilt, worthlessness, or helplessness; loss of interest in normally pleasurable activities; difficulty concentrating or with memory loss; restlessness or irritability; and sleeping too much or inability to sleep. A person with bipolar disorder is prone, in depressive episodes, to suicidal thoughts or even suicide attempts.

Without treatment, the natural course of bipolar disorder tends to worsen, with a person suffering more severe extremes of mood fluctuation, as well as a more frequent (faster-cycling) occurrence of mood swings. There appears to be at least some familial component to the disorder, as children of bipolar parents are at increased risk of developing the disease, as are identical twins where one twin has been diagnosed with bipolar disorder. Genetic studies suggest that multiple genes contribute to bipolar disorder.

Lithium, the first mood stabilizing medication approved in the United States by the U.S. Food and Drug Administration (FDA) for treatment of mania, is often very effective in controlling mania and preventing the recurrence, of both manic and depressive episodes. While other drugs, particularly anticonvulsants such as valproate (Depakote®) or carbamazepine (Tegretol®); benzodiazepines such as clonazepam (Klonopin®); antipsychotics such as clozapine (Clozaril®); or electroconvulsive (ECT) therapy are in use or under study for bipolar disorder, lithium remains a first line treatment. Accordingly, the art has sought ever improved methods of delivering pharmacologically effective dose, with minimum side effects, to sufferers of bipolar disorder.

Lithium has been used for medical purposes in various formulations for more than 150 years, although the modern use of lithium as an effective antimanic treatment and as prophylactic therapy for bipolar (manic-depressive) disorder dates to the early 1950's.

Lithium is abundant in some alkaline mineral spring waters and is present in trace amounts in animal tissues, although it has no known physiological role. Since its earliest use, lithium has been associated with potentially toxic effects, due both to its relative low therapeutic index, in the range of 2 or 3, and in part to the difficulty in achieving regulated dissolution and uptake in the human body. Both lithium carbonate and lithium citrate are currently in therapeutic use in the United States.

Lithium shares many of the physicochemical properties of the alkali metals group (Group Ia of the Periodic Table, which also includes sodium and potassium), of which it is the lightest member. It is a monovalent cation and has the highest electrical field density and largest energy of hydration of Group Ia, yet it has an ionic radius similar to those of the divalent cations magnesium and calcium. Lithium has a relatively small gradient of distribution across biological membranes, unlike sodium and potassium.

Therapeutic concentrations of lithium ion (Li⁺) have almost no discernible psychotropic effects in normal individuals. It is not a sedative, depressant, or euphoriant to normal individuals, and this lack of characteristic effects differentiate it from other psychotropic agents.

Li⁺ is absorbed readily and almost completely from the gastrointestinal tract, although rate of absorption is considerably affected by the type of formulation administered. Complete absorption occurs in about 8 hours, with peak concentrations in plasma occurring 2 to 4 hours after an oral dose. However, in certain formulations, absorption can occur considerably faster. For example, in a paper by Nielsen-Kudsk and Amdisen entitled, “Analysis of the Pharmacokinetics of Lithium in Man,” in European Journal of Clinical Pharmacology, 16, 271-77 (1979), a single liquefied dose of lithium chloride administered to volunteers in a pharmacokinetic study was shown to have a mean absorption half-time of only 0.15 hours (9 minutes).

Slow release formulations of lithium carbonate provide a slower rate of absorption and thereby minimize early peaks in plasma concentration of the Li⁺ ion. For example, in a paper entitled, “In vivo Evaluation of Two Controlled Release Lithium Carbonate Tablets,” in Lithium (1992) 3, 221-23, Gai, et al., reported on a formulation consisting of lithium carbonate, Avicel®, Lactose, Eudragit®(aqueous methacrylic polymer) and a lubricant (magnesium stearate) that demonstrated a slower lithium release when compared to a conventional formulation control. A commercial lithium carbonate dosage form is available from GlaxoSmithKline and is marketed as Eskalith CR 450®. The Eskalith CR 450® dosage form comprises:

| | | |
|---|---|---|
| Lithium Carbonate | 450 | mg |
| Iron Oxide | 1 | mg |
| Gelatin - 200 bloom | 40 | mg |
| Sodium Starch Glycolate | 0.75 | mg |
| Alginic Acid | 1 | mg |
| Magnesium Stearate | 5.25 | mg |
| TOTAL | 498 | mg |

The convenience of administering a single dose of a medication which releases active ingredients in a controlled fashion over an extended period of time, as opposed to the administration of a number of single doses at regular intervals, has long been recognized and desired in the pharmaceutical arts. The advantage to the patient and clinician in having consistent and uniform blood levels of medication over an extended period of time are likewise recognized. Among the most important advantages are: (1) increased contact time for the drug to allow for local activity in the stomach, intestine or other locus of activity; (2) increased and more efficient absorption for drugs which have specific absorption sites; (3) the ability to reduce the number of dosages per period of time; (4) employment of less total drug; (5) minimization or elimination of local and/or systemic side effects; (6) minimization of drug accumulation associated with chronic dosing; (7) improved efficiency and safety of treatment; (8) reduced fluctuation of drug level; and (9) better patient compliance with overall disease management.

The administration of slow release formulations is not without problems. These problems are particularly relevant in the case of lithium formulations. Slow release preparations tend to shift a higher percentage of total absorption into time periods further from administration, during which time the medication has further traversed the gastrointestinal tract, and therefore may cause a higher proportion of the lithium to be absorbed in the lower intestinal tract. This may cause such symptoms as nausea, vomiting, abdominal pain, and diarrhea. Li⁺ is poorly tolerated in as many as one third of all patients treated. Accordingly, the art has needed a means for maximizing the dissolution rate of lithium formulations in the intermediate time ranges for absorption, that is, within approximately three hours of administration.

Li⁺ is initially distributed in the extracellular fluid and then gradually accumulates in various human tissues. Passage through the blood-brain barrier is slow, and when a steady state is achieved, the final concentration of Li⁺ in the cerebrospinal fluid is about half or less of the concentration in plasma. In a study of both immediate release and controlled release dosages in a paper entitled “Absorption and disposition kinetics of lithium carbonate following administration of conventional and controlled release formulations,” in the International Journal of Clinical Pharmacology, Therapy and Toxicology, Vol. 24, 5:240-45 (1986), Arancibia, et al., found that a single oral dose of lithium confers upon the body the pharmacokinetic characteristics of an open two-compartment model with apparent first order absorption. The volume of distribution of lithium was found to be near the volume of the total body water with the volume of the central compartment approximately corresponding to the volume of the extracellular water.

Ninety-five percent of a single dose of Li⁺ is eliminated in the urine, with an initial excretion of one half to two thirds of an acute dose being excreted in a 6 to 12 hour period. This initial phase is followed by slow excretion over the next 10 to 14 days. Small amounts of Li⁺ are excreted in sweat and feces. The elimination half-life averages 20 to 24 hours, although any factor leading to Na⁺ depletion will tend to promote Li⁺ retention and thereby prolong the half-life of Li⁺. There is no specific treatment for Li⁺ overdose, which can manifest as mental confusion, hyperreflexia, gross tremor, dysarthria, seizures, cranial nerve and focal neurologic signs, and cardiac arrhythmias. These symptoms can progress to coma and death. Treatment is supportive, with maintenance of appropriate Na⁺ levels and hydration. Dialysis is the most effective means of removing Li⁺ from the body and is used in severe cases of lithium intoxication.

Accordingly, what the pharmaceutical arts have long sought is a means of formulating lithium that exhibits improved dissolution rates in an intermediate time period, as compared to presently available formulations. Formulations that are too quickly dissolved tend to create quick spikes in serum levels with an increased risk of toxicity, while those that are too slowly dissolved will tend to have a higher proportion of lithium absorbed in the lower gastrointestinal tract, leading to unpleasant symptoms and possible interference with patient compliance. Additionally, practical dispensing requirements mandate that commercial formulations have acceptable stability levels over time under a wide variety of storage conditions.

The purpose of the present invention is to replace a formulation and manufacturing process that is associated with poor control of dissolution rate with a formulation and process that is less complex to perform, more reproducible, and eliminates formulation and dissolution rate dependencies on raw material density and tablet press parameters. An example of current lithium carbonate (Eskalith CR 450®) product specifications, shown in Table 1, calls for dissolution rates of not more than 40% at 1 hour, 45-75% at three hours, and not less than 70% at seven hours.

TABLE 1
Eskalith CR 450 ® Product Dissolution Specifications

| Time | Dissolution % |
|---|---|
| 1 Hour | Not more than 40% |
| 3 Hour | 45%-75% |
| 7 Hour | Not less than 70% |

To this end, a series of experiments were undertaken to examine formulations of lithium carbonate combined with various excipients and formulated via different manufacturing techniques. The following materials were examined as sustained release agents for lithium carbonate and all of them failed:

Alginic Acid

Sodium Alginate

Guar Gum

Carbopol 971

Carbopol 974

Hydroxypropylmethylcellulose E4M

Hydroxypropylmethylcellulose E50

PVP K30

PVP K90

Hydroxypropylcellulose (solvent based)

Gelatin 125 bloom

Gelatin 150 bloom

Aquacoat (aqueous ethylcellulose)

Starch NF

Starch 1500 (partially pregelatinized starch)

Starch 1551 (totally pregelatinized starch)

Gelatin 200 bloom

Maltodextran M150

PEG4000

The only sustained release agent that merited further investigation was sodium carboxymethylcellulose.

### SUMMARY OF THE INVENTION

Drugs are seldom dispensed in pure form, instead they are commonly mixed with varying non-active agents, deemed excipients, to facilitate production, improve dispersal and dissolution characteristics, promote stability, and to increase palatability. Sodium carboxymethylcellulose (NaCMC) is known to be a stability and viscosity enhancer. It is widely used in oral and topical pharmaceutical preparations for its viscosity enhancing properties, to stabilize emulsions, and, as in the present invention, as a tablet binder and disintegrant. Chemically, NaCMC is the sodium salt of a polycarboxymethyl ether of cellulose, typically with a molecular weight in the range of 90,000-700,000. NaCMC is highly insoluble in organic solvents such as acetone, ethanol, ether, and toluene; but is easily dispersed in water at all temperatures. It is generally considered as a non-toxic and nonirritant material that is safe at a wide level of dosage. NaCMC has no acceptable daily intake level set by the World Health Organization, is listed as a substance that may be added to all foodstuffs in the European Council Directive No. 95/2/EC, and is included in the FDA Inactive Ingredients Guide. In doses exceeding 4 grams daily, NaCMC may have a bulk laxative effect due to its hygroscopic properties and ability to bind water during stool transit through the intestine.

As long ago as 1986, NaCMC was shown, by Arancibia et al., in concentrations of 30 percent, to slow an experimental rise seen in serum concentrations of lithium. A single dose NaCMC-lithium carbonate formulation, compared to a single dose immediate release preparation, showed a delay in the development of peak serum levels from less than two hours to approximately four hours. The effect of NaCMC formulations in altering the dissolution rates does not appear specific to lithium, as Singh demonstrated faster dissolution of lorazepam with NaCMC added, in a paper entitled, “Effect of Sodium Carboxymethylcellulose on the Disintegration, Dissolution, and Bioavailability of Lorazepam from Tablets,” in Drug Development and Industrial Pharmacy, 18(3): 375-83 (1992). There is evidence suggesting that the viscosity grade of NaCMC used may affect the balance of forces between those which hold the formulation particles together in tablet form and those which promote separation of the particles in water, as seen in a paper entitled, “Evaluation of different viscosity grades of sodium carboxymethylcellulose as tablet disintegrates, ” in Pharm. Acta Helv. 50(4): 99-102 (1975).

In the instant invention, the addition of a relatively small quantity (as compared to the prior art) of sodium carboxymethylcellulose to a formulation of lithium carbonate was found to enhance the dissolution profile of the formulation. Additionally, the utilization of a secondary release agent, glycine, was found to enhance dissolution rates. Furthermore, a manufacturing variation in which a portion of the active ingredient lithium carbonate was reserved from the initial granulation and then added later, with excipients, was found to enhance dissolution. Thus, there is disclosed a pharmaceutical composition for oral administration, comprising:

a. lithium carbonate,

b. optional pharmacologic excipients,

c. at least one dissolution rate stabilizer, and

d. at least one secondary release agent.

The pharmaceutical compositions according to the invention, may also contain iron oxide as a colorant. Preferably, the iron oxide does not exceed a level of about 1 mg/tablet. The pharmaceutical compositions according to the invention may also contain an optional pharmacological excipient, which may be a lubricant. The lubricants may be selected from the group consisting of a stearic acid at a concentration between about 0.1 percent and about 1 percent by weight of the composition, sodium sterol fumerate at a concentration of from about 0.1 to about 1.0 percent of the composition by weight, calcium stearate at a concentration of about 0.1 to 1.0 percent by weight of the composition, and magnesium stearate at a concentration of about 0.1 to 1.0 percent of the composition by weight.

The compositions according to the invention are preferably compressed in a conventional pharmaceutical tableting press at a tablet hardness of about 7 kPa to 20 kPa.

Most preferably, the pharmaceutical compositions according to the invention contain at least one dissolution rate stabilizer which is most preferably sodium carboxymethylcellulose. The sodium carboxymethylcellulose is usually present at about 5 to about 15 percent by weight of the composition. More preferably, the sodium carboxymethylcellulose comprises not more than about 5 percent by weight of the composition.

Still more preferably, the pharmaceutical composition according to the invention additionally comprises at least one secondary release agent and most preferably this secondary release agent is glycine. The glycine is typically present comprises between about 0.5 to about 40 mg/tablet.

Thus, there is further disclosed a pharmaceutical composition for oral administration, comprising:

a. lithium carbonate,

b. iron oxide,

c. stearic acid,

d. sodium carboxymethylcellulose,

e. glycine, and

f. optionally pharmaceutically acceptable excipients.

There is further disclosed a process for preparing controlled release solid dosage forms of lithium carbonate which comprises the steps of:

a. mixing lithium carbonate and iron oxide into a blend,

b. solubilizing a water solution of water, sodium carboxymethylcellulose, and at least one secondary release agent,

c. placing the blend of lithium carbonate and iron oxide in a bed of a fluid bed granulator,

d. creating a top sprayed blend by top spraying the solution into the blend in the bed of the fluid bed granulator,

e. granulating the top sprayed blend in the fluid bed granulator into a granulation,

f. forming a composition by milling the granulation with at least one excipient, and

g. pressing the granulation with at least one excipient composition into tablets in a tablet press.

The present invention also relates to methods of treatment of bipolar disorder consisting of orally administering to a patient a therapeutically effective amount of a composition in accordance with the invention. Another aspect of the present invention simply relates to the discovery that fairly low levels of carboxymethylcellulose are effective in preparing lithium carbonate dosage forms. Thus, there is disclosed a pharmaceutical composition for oral administration, comprising:

a. lithium carbonate,

b. optional pharmacological excipients, and

c. at least one dissolution rate stabilizer.

As discussed previously, iron oxide and lubricants may be present in the composition. The preferred dissolution rate stabilizer is sodium carboxymethylcellulose in this aspect of the invention wherein glycine is not required. Typically this sodium carboxymethylcellulose is present in a concentration from about 5 to about 15 percent by weight of the composition. Usually the lithium carbonate is present in a concentration of about 85 to 95 percent by weight of the composition.

Processes for the production of the pharmaceutical composition are as described above except the glycine would be omitted.

Yet another aspect of the present invention relates to the discovery that dividing the bowl charge will dramatically impact upon the stability and dissolution rates of the inventive dosage forms. Thus there is disclosed a process for manufacturing a pharmaceutical composition for oral administration, which comprises the steps of:

a. lithium carbonate,

b. optional pharmacological excipients, and

c. at least one dissolution rate stabilizer.

In a preferred embodiment, the pharmaceutical composition of the present invention has a dissolution profile as follows: 1 hour, no greater than 40 percent; at 3 hours, from 45 to 75 percent; and at 7 hours, not less than 70 percent. More preferably, the pharmaceutical composition of the present invention has a dissolution profile as follows: 1 hour, no greater than 40 percent; at 3 hours, from 50 to 65 percent; and at 7 hours, not less than 70 percent

### DETAILED DESCRIPTION OF THE INVENTION

Given the history of utility of using NaCMC and other excipients known in pharmaceutical manufacture with properties for alteration of drug dissolution rates, experiments were undertaken using lithium carbonate and NaCMC. The objectives of these experiments was to simplify the process, increase capacity, decrease batch variability, decrease batch failure rates, eliminate the formulation dependence on raw material density, eliminate the dependence on tablet press parameters as the release rate controlling factor, and to achieve acceptable stability.

For all experiments, a 45-liter granulation insert fluid bed granulator (GPCG) was charged with 16 to 18 kilograms of lithium carbonate with 1 mg/tablet of iron oxide added as a colorant. A release sustaining agent solution or suspension containing various release controlling agents was top sprayed in a volume of 20 to 40 kg onto the fluidized lithium carbonate and then dried. The granulation was milled with a GS 180 mill fitted with a 1.0 mm round hole cone. The milled granulation was blended in a PK 8-quart V blender with various extragranular ingredients, including a lubricant. Each blend was compressed using a Manesty 30 station high-speed commercial tablet press with 1.1 cm deep cup round punches.

The effect of modifying the type, amount, and ratio of the release sustaining agents and lubricants along with adjustments in compression were evaluated by examining the dissolution profile of the finished tablets. In an attempt to determine the robustness of the formulations, some studies were repeated, holding the excipients constant, utilizing different lots of lithium carbonate. The most promising formulations were placed on stability in glass and plastic packages.

### EXAMPLE I

### Lithium Carbonate—NaCMC Formulations

To test the dissolution rates of lithium carbonate with NaCMC, and stability of these rates; tests of potency, dissolution, and stability were performed on lithium carbonate formulated with varying amounts of added NaCMC, as shown in Table 2. There was some variation seen in loss on drying (LOD). The addition of approximately 10%, or 51.07 mg, added NaCMC resulted in an improved dissolution at the three hour point, and the addition of further NaCMC did not appreciably improve this dissolution rate, as shown in Table 2. Therefore, a level of approximately 10% added NaCMC was selected for further experimentation. Additionally, the effects on dissolution of varying the levels of stearic acid lubricant in a lithium carbonate/iron oxide formulation were observed, as shown in Tables 3 and 4, and found to have little effect on dissolution. However, it was found from a manufacturing standpoint that a stearic acid level of approximately 1%, or 5.07 mg/tablet, was found to give optimal results in tablet appearance and consistency, and this level was selected for further experimentation. Various combinations of other lubricants at differing concentrations showed no improvement in dissolution parameters when compared to the use of stearic acid as a lubricant, as shown in Tables 4-7.

TABLE 2
Effects of Varying Levels of NaCMC on Dissolution

| | | Stearic | Tablet | 1 Hour | 3 Hour | 7 Hour | |
|---|---|---|---|---|---|---|---|
| | NaCMC | Acid | Weight | Dissolution | Dissolution | Dissolution | |
| Formula | mg/tablet | mg/tablet | Mg | % | % | % | % LOD |
| 3892 | 25.54 | 4.79 | 481.33 | 25 | 60 | 102* | 0.9 |
| 3891 | 51.07 | 4.79 | 506.86 | 23 | 59 | 99 | 0.9 |
| 5037 | 51.07 | 4.79 | 506.86 | 23 | 59 | 101* | 0.6 |
| 3893 | 76.61 | 4.79 | 532.4 | 21 | 62 | 95 | 1.7 |
| 100115 | 51.07 | 5.07 | 507.14 | 24 | 60 | 107* | 0.69 |

*percentage dissolution referenced to a baseline of 450 mg lithium carbonate per tablet. For tablet formulations exceeding this weight, calculated percentage greater than 100 percent is therefore possible.

TABLE 3
Effects of Varied Levels of Stearic Acid on Dissolution in NaCMC Granulation

| | | Stearic | Tablet | 1 Hour | 3 Hour | 7 Hour | |
|---|---|---|---|---|---|---|---|
| | NaCMC | Acid | Weight | Dissolution | Dissolution | Dissolution | |
| Formula | mg/tablet | mg/tablet | Mg | % | % | % | % LOD |
| 100115 | 51.07 | 5.07 | 507.14 | 24 | 60 | 107* | 0.69 |
| 100116 | 51.07 | 2.5 | 504.57 | 28 | 73 | 114* | 0.7 |
| 3891 | 51.07 | 4.79 | 506.86 | 23 | 59 | 99 | 0.9 |
| 5037 | 51.07 | 4.79 | 506.86 | 23 | 59 | 101* | 0.6 |
| 5495 | 51.07 | 5.07 | 507.14 | 23 | 53 | 95 | 1.2 |

*percentage dissolution referenced to a baseline or 450 mg lithium carbonate per tablet. For tablet formulations exceeding this weight, calculated percentage greater than 100 percent is therefore possible.

TABLE 4
Effect on Dissolution of NaCMC Granulation Utilizing
Varying Levels of Stearic Acid as Lubricant

| | Stearic | | Tablet | | 1 Hour | 3 Hour | 7 Hour |
|---|---|---|---|---|---|---|---|
| | Acid | Hardness | at 7 kPa | | Dissolution | Dissolution | Dissolution |
| Formula | Level | (kPa) | Hardness | Hardness | % | % | % |
| 0105327 | 1.0% | 7 | Porous | 17.2 | 19 | 51 | 93 |
| 0105326 | 0.5% | 7 | Porous | 18.0 | NA | NA | NA |
| 0105325 | 0.25% | 7 | Porous | 17.3 | 20 | 52 | 95 |
| 0105337 | 0.1% | 7 | * | * | * | * | * |
| 0105340 | 0.05% | 7 | Poor | 16.2 | NA | NA | NA |

* No sample
NA = Dissolution not performed, therefore data not available

TABLE 5
Effects on NaCMC Granulation Dissolution Varying Sodium Stearyl Fumerate as Lubricant

| | Sodium | | | | | | |
|---|---|---|---|---|---|---|---|
| | Stearyl | | Tablet | | 1 Hour | 3 Hour | 7 Hour |
| | Fumerate | Hardness | at 7 kPa | | Dissolution | Dissolution | Dissolution |
| Formula | Level | (kPa) | Hardness | Hardness | % | % | % |
| 0105327 | 1.0% | 7 | Porous | 19.3 | 20 | 51 | 92 |
| 0105329 | 0.5% | 7 | Porous | 22.0 | NA | NA | NA |
| 0105328 | 0.25% | 7 | Porous | 18.5 | 21 | 52 | 94 |
| 0105335 | 0.1% | 7 | Porous | 22.1 | NA | NA | NA |
| 0105336 | 0.05% | 7 | Poor | 18.5 | 20 | 51 | 94 |

NA = Dissolution not performed, therefore data not available

TABLE 6
Effects on NaCMC Granulation Dissolution Varying Calcium Stearate as Lubricant

| | Calcium | | Tablet at | | 1 Hour | 3 Hour | 7 Hour |
|---|---|---|---|---|---|---|---|
| | Stearate | Hardness | 7 kPa | | Dissolution | Dissolution | Dissolution |
| Formula | Level | (kPa) | Hardness | Hardness | % | % | % |
| 0105343 | 1.0% | 7 | Porous | 17.9 | 19 | 43 | 76 |
| 0105342 | 0.5% | 7 | Porous | 17.3 | NA | NA | NA |
| 0105341 | 0.25% | 7 | Porous | 16.6 | 20 | 47 | 90 |
| 0105344 | 0.1% | 7 | Porous | 12.6 | NA | NA | NA |
| 0105345 | 0.05% | 7 | Poor | 19.5 | NA | NA | NA |

NA = Dissolution not performed, therefore data not available

TABLE 7
Effects on NaCMC Granulation Dissolution Varying Magnesium Stearate as Lubricant

| | Magnesium | | Tablet at | | 1 Hour | 3 Hour | 7 Hour |
|---|---|---|---|---|---|---|---|
| | Stearate | | 7 kPa | | Dissolution | Dissolution | Dissolution |
| Formula | Level | Hardness | Hardness | Hardness | % | % | % |
| 0105332 | 1.0% | 7 | Porous | 19.6 | 18 | 43 | 79 |
| 0105331 | 0.5% | 7 | Porous | 23.7 | NA | NA | NA |
| 0105334 | 0.25% | 7 | Porous | 22.7 | 20 | 47 | 86 |
| 0105339 | 0.1% | 7 | Fair | 23.0 | NA | NA | NA |
| 0105338 | 0.05% | 7 | Poor | 19.2 | NA | NA | NA |

NA = Dissolution not performed, therefore data not available

The use of NaCMC in the formulation showed great promise, as these formulations exhibited desirable dissolution characteristics (between approximately 50% to 55% released at the three hour time point). These formulations were also shown to be the most robust with respect to dissolution, with little or no change in the dissolution rates occurring even when multiple parameters, such as relative percentage composition of NaCMC and stearic acid were changed, as shown in Tables 2 and 3. Various lubricant modifications and alterations in tablet hardness resulted in little change in dissolution rates, as shown in Tables 4 through 7.

In sum, when using NaCMC as the sole release sustaining agent, the following observations were made from the material presented in Tables 4-7:

1. Increasing the level of NaCMC beyond 5.3% produced no effect on the dissolution rate.

2. Processing several blends and compressions from the same lot of granulation (using a single lot of lithium carbonate) produced no change in the dissolution rate.

3. The use of multiple lots of lithium carbonate (for granulation) produced no change in the dissolution rate.

4. The compression rate of tablets at multiple hardness levels (7, 10, or approximately 20 kPa) produced no change in the dissolution rate.

5. Modifying the level (0.05 to 1.0%) or type of lubricant did not significantly alter the release rate.

Stability testing performed in both glass bottles and in the current commercial packaging, however, showed a decrease in dissolution rates of the lithium carbonate-NaCMC formulations over time, as shown in Table 8. At higher temperature, higher relative humidity, and longer storage times, the lithium carbonate-NaCMC formulations tended to fall close to, or even outside of, current product specifications for the three hour dissolution period, which call for a dissolution rate of 45% to 75% within three hours, as shown in Table 1.

This decrease in dissolution was seen both when testing in the current commercial packaging, and in glass bottles, as seen in Tables 8 and 9. It was hypothesized that a modest improvement in the initial dissolution rates, possibly to approximately the high 50% to low 60% range, would provide a margin for the observed deterioration in dissolution rates over time, and allow new formulations demonstrating acceptable dissolution rates both upon manufacture, and after storage.

In attempts to overcome this loss of dissolution stability over time, multiple experiments were undertaken with a goal to enhance the release rate ranging from approximately 60% to approximately 65% in the three hour time period, in order to provide a margin for the observed loss of dissolution stability at longer storage periods, which caused the NaCMC formulation to fall outside of specifications at the six month, higher temperature and higher relative humidity conditions, as shown in Tables 8 and 9.

TABLE 8
Dissolution Stability Summary, NaCMC - Lithium (Current Commercial Packaging)

| | | Potency | 1 Hour | | | 3 Hour | | | 7 Hour | | |
|---|---|---|---|---|---|---|---|---|---|---|---|
| Storage | Age | (% of | Dissolution % | | | Dissolution % | | | Dissolution % | | |
| Conditions | (Months) | Claimed) | High | Low | Avg. | High | Low | Avg. | High | Low | Avg. |
| Initial | 00 | 98.8 | 21 | 20 | 20 | 52 | 49 | 51 | 94 | 91 | 93 |
| 25° C./60% | 01 | NR | 22 | 21 | 21 | 55 | 48 | 53 | 97 | 92 | 94 |
| Rel. Hum. | | | | | | | | | | | |
| | 02 | NR | 22 | 21 | 21 | 55 | 48 | 53 | 97 | 92 | 94 |
| | 03 | 99.4 | 20 | 19 | 19 | 52 | 49 | 50 | 95 | 87 | 90 |
| | 04 | 100.2 | 21 | 19 | 20 | 52 | 49 | 50 | 95 | 87 | 90 |
| | 05 | 98.2 | 19 | ]8 | 18 | 51 | 49 | 50 | 93 | 87 | 90 |
| | 06 | 98.7 | 20 | 19 | 19 | 50 | 48 | 49 | 91 | 86 | 88 |
| | 09 | | 18 | 17 | 18 | 48 | 46 | 47 | 81 | 87 | 89 |
| 30° C./60% | 03 | NR | NR | NR | NR | NR | NR | NR | NR | NR | NR |
| Rel. Hum. | | | | | | | | | | | |
| | 05 | 98.9 | 19 | 17 | 18 | 51 | 49 | 49 | 93 | 87 | 90 |
| | 06 | 98.2 | 20 | 16 | 18 | 50 | 44 | 47 | 93 | 83 | 88 |
| | 09 | | 19 | 18 | 18 | 48 | 47 | 47 | 87 | 85 | 86 |
| 40° C./75% | 01 | NR | 21 | 20 | 20 | 52 | 50 | 51 | 94 | 90 | 92 |
| Rel. Hum. | | | | | | | | | | | |
| | 02 | NR | 19 | 19 | 18 | 51 | 48 | 50 | 93 | 89 | 91 |
| | 03 | 99.5 | 19 | 19 | 18 | 51 | 48 | 50 | 93 | 89 | 91 |
| | 04 | 99.5 | 17 | 16 | 17 | 44 | 40 | 42 | 81 | 76 | 79 |
| | 06 | 96.9 | 17 | 15 | 16 | 44 | 40 | 41 | 82 | 75 | 79 |
| Spec. Limits | | 99.0-110.0 | Not | More | Than | 45% | - | 75% | Not | Less | Than |
| | | | | 40% | | | | | | 75% | |

TABLE 9
Stability Summary, Lithium Carbonate - NaCMC (Glass Bottles)

| | | | 1 Hour | | | 3 Hour | | | 7 Hour | | |
|---|---|---|---|---|---|---|---|---|---|---|---|
| Storage | Age | | Dissolution % | | | Dissolution % | | | Dissolution % | | |
| Conditions | (Months) | Potency | High | Low | Avg. | High | Low | Avg. | High | Low | Avg. |
| Initial | 00 | 99.5 | 20 | 20 | 20 | 52 | 50 | 51 | 93 | 89 | 92 |
| 25° C./60% | 01 | NR | 22 | 20 | 21 | 50 | 53 | 52 | 97 | 92 | 94 |
| Rel. Hum. | | | | | | | | | | | |
| | 02 | NR | 20 | 19 | 20 | 52 | 50 | 51 | 93 | 89 | 91 |
| | 03 | 99.2 | 22 | 18 | 20 | 54 | 49 | 51 | 94 | 91 | 93 |
| | 04 | 98.8 | 20 | 19 | 20 | 51 | 49 | 50 | 94 | 87 | 91 |
| | 05 | 98.4 | 19 | 18 | 19 | 50 | 48 | 49 | 91 | 88 | 89 |
| | 06 | 97.6 | 23 | 21 | 23 | 52 | 48 | 50 | 89 | 84 | 87 |
| | 09 | | 17 | 16 | 17 | 49 | 46 | 48 | 91 | 86 | 89 |
| 30° C./60% | 03 | NR | NR | NR | NR | NR | NR | NR | NR | NR | NR |
| Rel. Hum. | | | | | | | | | | | |
| | 05 | 98.5 | 20 | 18 | 19 | 49 | 48 | 48 | 89 | 85 | 87 |
| | 06 | 98.0 | 20 | 16 | 18 | 50 | 44 | 47 | 93 | 84 | 88 |
| | 09 | | 20 | 18 | 19 | 50 | 47 | 49 | 92 | 87 | 91 |
| 40° C./75% | 01 | NR | 20 | 19 | 20 | 52 | 50 | 51 | 96 | 84 | 92 |
| Rel. Hum. | | | | | | | | | | | |
| | 02 | NR | 19 | 18 | 18 | 50 | 48 | 48 | 94 | 87 | 88 |
| | 03 | 100.0 | 19 | 18 | 19 | 49 | 44 | 46 | 88 | 81 | 84 |
| | 04 | 99.2 | 18 | 17 | 17 | 49 | 44 | 46 | 88 | 81 | 84 |
| | 06 | 98.4 | 15 | 17 | 16 | 44 | 40 | 42 | 83 | 77 | 80 |
| Spec. Limits | | 99.0-110.0 | Not | More | Than | 45% | - | 75% | Not | Less | Than |
| | | | | 40% | | | | | | 75% | |

NR = Not Required

### EXAMPLE II

### Lithium Carbonate—NaCMC with Additional Excipients

Attempts were made to improve three hour dissolution rates using aqueous NaCMC as the release sustaining agent combined with other extragranular excipients. NaCMC was dissolved in water and used to granulate the lithium carbonate/iron oxide blend. The granulation was milled in a cone mill fitted with a 1 mm round hole cone. Prior to lubrication, the granulation was blended with additional excipients including multiple levels of Aerosil 200® (colloidal silica), Avicel PH102® (microcrystalline cellulose), Starch 1500 (partially gelatinized starch), and/or lactose. The blend was then lubricated and compressed at multiple hardness levels (7 and 10 kPa). Experimental variables included the manufacture of batches using different lots of lithium carbonate, the manufacture of multiple batches from a single lot of lithium carbonate, the manufacture of multiple batches of tablets from a single batch of granulation, the manufacture of batches with different levels of NaCMC, the manufacture of batches with different levels of Aerosil 200®, the manufacture of batches with different levels of Starch 1500, the manufacture of batches with different levels of Avicel PH102®, the manufacture of batches with different levels of lactose, and the manufacture of batches using different levels of lubricant, utilizing either stearic acid or magnesium stearate.

The extragranular addition of these materials to the lithium carbonate/iron oxide/NaCMC formulation reduced the dissolution rate to low borderline or below (45%) for the three hour time point, as shown in Tables 10-13

TABLE 10
Effects on Dissolution of Avicel ® in NaCMC Granulation

| | | | Starch | Aerosil | Stearic | | | | | |
|---|---|---|---|---|---|---|---|---|---|---|
| | NaCMC | Avicel ® | 1500 | 200 ® | Acid | Tab wt. | Dissolution % | | | |
| Formula | mg/tablet | mg/tablet | mg/tablet | Mg/tablet | mg/tablet | mg | 1 Hr. | 3 Hr. | 7 Hr. | % LOD |
| 5499 | 51.07 | 0 | 25 | 6 | 5.4 | 538.45 | 19 | 51 | 92 | .8 |
| 5498 | 51.07 | 10 | 25 | 6 | 5.5 | 548.56 | 20 | 49 | 93 | .3 |
| 4519 | 51.07 | 15 | 25 | 6 | 5.5 | 553.61 | 18 | 45 | 88 | .1 |
| 5035 | 51.07 | 15 | 25 | 6 | 5.5 | 553.61 | 17 | 41 | 74 | .1 |

TABLE 11
Effects on Dissolution of Starch 1500 and Lithium Lot in NaCMC Granulations

| | | | Starch | Stearic | | | | | | | |
|---|---|---|---|---|---|---|---|---|---|---|---|
| | NaCMC | Avicel ® | 1500 | Acid | Tab Wt. | Dissolution % | | | | Lithium | Granulation |
| Formula | mg/tablet | Mg/Tablet | mg/tablet | mg/tablet | Mg | 1 Hr. | 3 Hr. | 7 Hr. | % LOD | Lot | Batch # |
| 100106 | 51.07 | 15 | 45 | 5.7 | 573.8 | 14 | 40 | 83 | 0.84 | 2632 | 6265 |
| 100111 | 51.07 | 15 | 45 | 5.7 | 573.81 | 17 | 48 | 89 | 0.8 | 2632 | 6266 |
| 100104 | 51.07 | 15 | 35 | 5.6 | 563.7 | 14 | 39 | 77 | 0.84 | 2632 | 6265 |
| 100103 | 51.07 | 15 | 25 | 5.5 | 553.6 | 15 | 42 | 79 | 0.84 | 2632 | 6265 |
| 100107 | 51.07 | 15 | 25 | 5.5 | 553.6 | 19 | 49 | 90 | 0.83 | 2632 | 6266 |
| 100114 | 51.07 | 15 | 25 | 5.5 | 553.61 | 18 | 51 | 92 | 0.74 | 2603 | 6264 |
| 100106 | 51.07 | 15 | 45 | 5.7 | 573.8 | 14 | 40 | 83 | 0.84 | 2632 | 6265 |
| 100112 | 51.07 | 15 | 45 | 5.7 | 573.81 | 17 | 47 | 87 | 0.81 | 2603 | 6264 |
| 100110 | 51.07 | 15 | 15 | 5.4 | 543.51 | 20 | 52 | 97 | 0.75 | 2632 | 6266 |
| 100113 | 51.07 | 15 | 15 | 5.4 | 543.51 | 18 | 51 | 91 | 0.71 | 2603 | 6264 |

TABLE 12
Effects on Dissolution of Aerosil 200 ® in NaCMC Granulations

| | | | Starch | Aerosil | Stearic | | | | | |
|---|---|---|---|---|---|---|---|---|---|---|
| | NaCMC | Avicel ® | 1500 | 200 ® | Acid | Tab Wt. | Dissolution % | | | |
| Formula | mg/tablet | mg/tablet | mg/tablet | Mg/tablet | mg/tablet | mg | 1 Hr. | 3 Hr. | 7 Hr. | % LOD |
| 5497 | 51.07 | 15 | 25 | 0 | 5.48 | 547.55 | 19 | 48 | 91 | 2.2 |
| 4520 | 51.07 | 15 | 25 | 4 | 5.51 | 551.58 | 18 | 49 | 92 | 1.2 |
| 5036 | 51.07 | 15 | 25 | 4 | 5.52 | 551.59 | 18 | 46 | 85 | 1.0 |
| 5496 | 51.07 | 15 | 25 | 4 | 5.52 | 551.59 | 18 | 48 | 88 | 2.1 |
| 5035 | 51.07 | 15 | 25 | 6 | 5.54 | 553.61 | 17 | 41 | 74 | 1.1 |
| 5034 | 51.07 | 15 | 25 | 8 | 5.56 | 555.63 | 16 | 44 | 82 | 1.3 |
| 5038 | 51.07 | 15 | 25 | 8 | 5.56 | 555.63 | 15 | 43 | 81 | 0.8 |
| 4518 | 51.07 | 15 | 25 | 10 | 5.58 | 557.65 | 18 | 45 | 91 | 1.3 |
| 5033 | 51.07 | 15 | 25 | 10 | 5.58 | 557.65 | 17 | 41 | 74 | 0.9 |

TABLE 13
Effect of Lactose Replacement of Avicel ® on Dissolution of NaCMC Granulations

| | | | Starch | Aerosil | Stearic | | | | | |
|---|---|---|---|---|---|---|---|---|---|---|
| | NaCMC | Lactose | 1500 | 200 ® | Acid | Tab Wt. | Dissolution % | | | |
| Formula | mg/tablet | mg/tablet | mg/tablet | Mg/tablet | mg/tablet | mg | 1 Hr. | 3 Hr. | 7 Hr. | % LOD |
| 101438 | 51.07 | 5 | 25 | 6 | 5.44 | 543.51 | 15 | 43 | 85 | 0.63 |
| 101442 | 51.07 | 15 | 25 | 6 | 5.54 | 553.61 | 16 | 44 | 87 | 0.60 |
| 101440 | 51.07 | 45 | 25 | 6 | 5.84 | 583.91 | 16 | 44 | 84 | 0.62 |

Variations in stearic acid lubricant concentrations were also tested with representative formulations containing NaCMC, Avicel®, Starch 1500, and Aerosil 200®. Experimentations in tablet compression revealed that the compression of tablets to pressures of either 7 kPa or 10 kPa produced no change in the dissolution rates.

TABLE 14
Effects on Dissolution of Varying Levels of Stearic Acid Lubricant in Representative
NaCMC Granulations Containing Avicel ®, Starch 1500, and Aerosil 200 ®

| | | | Starch | Aerosil | Stearic | | | | | |
|---|---|---|---|---|---|---|---|---|---|---|
| | NaCMC | Avicel ® | 1500 | 200 ® | Acid | Tab Wt. | Dissolution % | | | |
| Formula | mg/tablet | Mg/tablet | mg/tablet | mg/tablet | mg/tablet | mg | 1 Hr. | 3 Hr. | 7 Hr. | % LOD |
| 100109 | 51.07 | 15 | 25 | 6 | 8.35 | 556.42 | 18 | 47 | 89 | 0.8 |
| 5496 | 51.07 | 15 | 25 | 4 | 5.52 | 551.59 | 18 | 46 | 88 | 2.1 |
| 100107 | 51.07 | 15 | 25 | 6 | 5.53 | 553.6 | 19 | 49 | 90 | 0.83 |
| 6296 | 51.07 | 15 | 25 | 4 | 4.1 | 550.17 | 21 | 51 | 94 | 1.7 |
| 6295 | 51.07 | 15 | 25 | 4 | 2.75 | 548.82 | 21 | 53 | 98 | 1.5 |
| 100108 | 51.07 | 15 | 25 | 6 | 2.8 | 550.87 | 19 | 54 | 100 | 0.76 |

The following observations were made from the results presented in Tables 10-14:

1. Modification in the levels of Aerosil® produced no change in the dissolution rate.

2. Modification in the levels of starch produced no change in the dissolution rate.

3. An increase in the level of Avicel®0 produced aminor slowing of the dissolution rate.

4. Replacement of Avicel® with lactose produced no change in the dissolution rate.

5. Modification in the levels of lactose produced no change in the dissolution rate.

6. Processing several blends and compressions from the same lot of granulation, using a single lot of lithium carbonate, produced no change in the dissolution rate.

7. The use of multiple lots of lithium carbonate for different batches of granulation produced no change in the dissolution rate.

8. A reduction of ⁵⁰% in the level of lubricant produced aminor increase in dissolution.

9. A 50% increase in the lubricant level did not produce a slowing effect on the dissolution rate.

### EXAMPLE III

### Replacement of NaCMC With Other Binding Agents

To test the efficacy of other binding agents in improving the dissolution characteristics of lithium carbonate, NaCMC was replaced with various other release sustaining agents, including starch, gelatin, aqueous polyvinylpyrrolidone (PVP), and hydroxypropylcellulose (HPC). Starch NF, Starch 1500 (partially pregelatinized starch), or Starch 1551 (totally pregelatinized starch) was suspended in water and used in place of NaCMC to granulate the lithium carbonate/iron oxide blend. The granulation was milled in a cone mill fitted with a 1 mm round hole cone. Prior to the lubrication the granulations were blended with additional excipients including multiple levels of Aerosil 200® (colloidal silica), Avicel PH102® (microcrystalline cellulose), and/or Starch 1500 (partially pregelatinized starch). The blends were then lubricated and compressed at hardness levels of either 7 or 10 kPa. With starch as the release sustaining agent, with or without additional extragranular excipients, the dissolution rates were found to be highly erratic and all work was stopped on these formulations.

In a second series of experiments, gelatin (Gelatin A, 125 and 200 bloom, or Gelatin B, 200 bloom) was suspended/dissolved in water and used to granulate the lithium carbonate/iron oxide blend. The granulation was milled in a cone mill fitted with a 1 mm round hole cone. In a sub-series of experiments using gelatin, the granulation was blended, prior to lubrication, with additional excipients, including multiple levels of Aerosil 200® (colloidal silica), Avicel PH102® (microcrystalline cellulose), Starch 1500 (partially gelatinized starch), Explotab (sodium starch glycolate), and/or Ac-Di-Sol (Croscarmellose sodium). The blends were then lubricated and compressed at hardness levels of either 7 or 10 kPa. Variations using different lots of lithium carbonate, the manufacture of multiple batches from a single lot of lithium carbonate, the manufacture of multiple batches or tablets from a single batch of granulation and differing levels of both stearic acid and magnesium stearate lubricant were all tested. When using gelatin as the release agent, with or without additional extragranular excipients, the dissolution rates were very erratic and all work was stopped on these formulations.

In a third series of experiments, polyvinylpyrrolidone (PVP), K30, K90, and K30 plus K90 (difference in molecular weights) was dissolved in water and used to granulate the lithium carbonate/iron oxide blends. The granulation was milled in a cone mill fitted with a 1 mm round hole cone. In a sub-series of experiments using polyvinylpyrrolidone, the granulation was blended, prior to lubrication, with additional excipients, including multiple levels of Aerosil 2000 (colloidal silica), Avicel PH102® (microcrystalline cellulose), and/or Starch 1500 (partially gelatinized starch). The blend was then lubricated and compressed at hardness levels of either 7 or 10 kPa. Variations including the manufacture of multiple batches from a single lot of lithium carbonate, the manufacture of multiple batches of tablets from a single granulation batch, the manufacture of batches with different levels and types of PVP and the manufacture of batches using different levels of both stearic acid and magnesium stearate lubricants were tested. When using PVP as the release sustaining agent, with or without extragranular excipients, the dissolution rates were highly erratic and all work was stopped on these formulations.

In a fourth series of experiments, hydroxypropylcellulose (HPC) was dissolved in an organic solvent (isopropyl alcohol) and used to granulate the lithium carbonate/iron oxide blend in an attempt to eliminate the use of water in the formulation process. The granulation was milled using a cone mill fitted with a 1 mm round hole cone. In a sub-series of experiments using HPC, the granulation was blended, prior to lubrication, with additional excipients including multiple levels of Aerosil 200® (colloidal silica), Avicel PH102® (microcrystalline cellulose), and/or Starch 1500 (partially gelatinized starch). The blend was then lubricated and compressed to hardness levels of either 7 or 10 kPa. Varying levels of stearic acid lubricant were tested. When using HPC as the release sustaining agent, with or without additional extragranular excipients, the dissolution rates were very fast and all work was stopped on these formulations.

### EXAMPLE IV

### Lithium Carbonate—NaCMC With Secondary Release Agent (Glycine)

With the failure of other release sustaining agents to improve the dissolution profile obtained with NaCMC as the release sustaining agent, experiments were undertaken utilizing a secondary release sustaining agent, glycine, in addition to the formulations including NaCMC. Multiple levels of glycine were used in conjunction with lithium carbonate/iron oxide. NaCMC formulations and release rates were found to be modified in a controllable manner, ranging from three hour dissolution rates of approximately 50% with no added glycine, and ranging upwards to three hour dissolution rates of nearly 100% with 40 mg/tablet added glycine, as shown in Table 15. As the goal of the experimental protocol was to achieve only a modest increase in the baseline dissolution rate using NaCMC as the sole release sustaining agent (see, e.g., three hour dissolution rate for Na-CMC in Tables 8 and 9), a level of 8 mg/tablet of added glycine, which produced an increase in the three hour dissolution rate to 63%, as seen in Table 15, was chosen for additional inquiry.

TABLE 15
Effects of Varying Levels of Glycine as Secondary
Releasing Agent in NaCMC Granulations

| | | Glycine | | Stearic | | Dissolution % | | |
|---|---|---|---|---|---|---|---|---|
| Formula | NaCMC | Level | | Acid | | 1 Hr. | 3 Hr. | 7 Hr. |
| 0106033 | 25 mg | 0.5 | mg | 2.4 | mg | 22 | 57 | 90 |
| 0106977 | 25 mg | 2 | mg | 2.4 | mg | 22 | 57 | 96 |
| 0106976 | 25 mg | 5 | mg | 2.4 | mg | 23 | 59 | 99 |
| 0106975 | 25 mg | 8 | mg | 2.45 | mg | 25 | 63 | 100 |
| 0107229 | 25 mg | 8 | mg | 4.9 | mg | 26 | 63 | 100 |
| 0107232 | 25 mg | 11 | mg | 4.9 | mg | 25 | 63 | 100 |
| 0107230 | 25 mg | 14 | mg | 4.9 | mg | 28 | 67 | 100 |
| 0106774 | 25 mg | 20 | mg | 2.5 | mg | 30 | 78 | 101* |
| 0106772 | 25 mg | 40 | mg | 2.6 | mg | 35 | 96 | 101* |

(*percentage dissolution referenced to a baseline of 450 mg lithium carbonate per tablet. For tablet formulations exceeding this weight, calculated percentage greater than 100 percent are therefore possible.)

| Formula | Tablet Weights (mg) |
|---|---|
| 0106033 | 477.9 |
| 0106977 | 479.4 |
| 0106976 | 482.4 |
| 0106975 | 485.45 |
| 0107229 | 487.9 |
| 0107232 | 490.9 |
| 0107230 | 493.9 |
| 0106774 | 497.5 |
| 0106772 | 517.6 |

In a preferred embodiment, the glycine ranges from, at a lower end, about 0.1, 1, 2, or 3 percent to about, at a higher end, 6, 8, 9, or 10 percent based on the weight of the composition.

Several other experiments were performed using this combination release controlling formulation. It was found, as shown in Table 16, that the density of the lithium carbonate did not affect the release profile. It was also found, as shown in Table 17, that increasing the lubricant level, utilizing either stearic acid or sodium stearyl fumerate, did slow the release rate. During these experiments the level of stearic acid was increased from 0.5% to 1.0% because although the tablets showed no dissolution problems at a 0.5% stearic acid level, the tablet tooling showed a relative lack of tablet lubricant. The increase in the level of stearic acid from 0.5% to 1.0% did not affect the dissolution profiles, however, levels above 1% did cause some slowing of dissolution, as shown in Tables 16 and 17.

TABLE 16
Effect of Different Lithium Bulk Densities on NaCMC-Glycine Granulations

| | | | Glycine | Stearic | | | |
|---|---|---|---|---|---|---|---|
| | NaCMC | Lithium | Level | Acid | Dissolution % | | |
| Formula | mg/tablet | Density | Mg/tablet | mg/tablet | 1 Hr. | 3 Hr. | 7 Hr. |
| 0200578 | 25 mg | 0.49 | 8 mg | 4.9 mg | 23 | 61 | 97 |
| 0200575 | 25 mg | 0.51 | 8 mg | 4.9 mg | 24 | 60 | 98 |
| 0200581 | 25 mg | 0.525 | 8 mg | 4.9 mg | 25 | 61 | 100 |
| 0200756 | 25 mg | 0.55 | 8 mg | 4.9 mg | 33 | 70 | 101* |
| 0106975 | 25 mg | 0.55 | 8 mg | 4.9 mg | 28 | 64 | 100 |

(*percentage dissolution referenced to a baseline of 450 mg lithium carbonate per tablet. For tablet formulations exceeding this weight, calculated percentage greater than 100 percent is therefore possible.)

TABLE 17
Effect of Increasing Lubrication on NaCMC-Glycine Granulations

| | | | Sodium | | | | | | |
|---|---|---|---|---|---|---|---|---|---|
| | | Glycine | Stearyl | | Stearic | | | | |
| | NaCMC | Level | Fumerate | | Acid | | Dissolution % | | |
| Formula | mg/tablet | mg/tablet | Mg/tablet | | mg/tablet | | 1 Hr. | 3 Hr. | 7 Hr. |
| 0200586 | 25 mg | 8 mg | 0 | mg | 4.9 | mg | 33 | 70 | 100 |
| 0200771 | 25 mg | 8 mg | 0 | mg | 9.8 | mg | 26 | 57 | 96 |
| 0200770 | 25 mg | 8 mg | 0 | mg | 14.9 | mg | 23 | 51 | 86 |
| 0200773 | 25 mg | 8 mg | 4.9 | mg | 0 | mg | 26 | 62 | 102* |
| 0200774 | 25 mg | 8 mg | 9.8 | mg | 0 | mg | 25 | 57 | 95 |

(*percentage dissolution referenced to a baseline of 450 mg lithium carbonate per tablet. For tablet formulations exceeding this weight, calculated percentage greater than 100 percent is therefore possible.)

Experiments were performed utilizing different levels of moisture and demonstrated that when drying the granulation, it is very difficult to dry to a moisture level much lower than 0.5% to 1.0%. A single compression was attempted with granulation at a 4.25% moisture level. The compression was only able to achieve a 4.0 kPa hardness level. This, however, did give a respectable dissolution rate of 60% at the three hour point.

Dissolution stability studies were undertaken for both low density and high density lithium, utilizing the lithium carbonate—NaCMC—Glycine formulation, as seen in Tables 18 and 19. These showed an improved dissolution rate compared to the dissolution stabilities seen with the lithium carbonate—NaCMC formulations, in the current commercial packaging, reported in Table 8.

TABLE 18
Dissolution Stability Summary Lithium Carbonate - NaCMC -Glycine Formulations
Utilizing Low Density Lithium in Current Commercial Packaging

| | | 1 Hour | | | 3 Hour | | | 7 Hour | | |
|---|---|---|---|---|---|---|---|---|---|---|
| Storage | Age | Dissolution % | | | Dissolution % | | | Dissolution % | | |
| Conditions | (Months) | High | Low | Avg. | High | Low | Avg. | High | Low | Avg. |
| Initial | 00 | 26 | 25 | 26 | 61 | 58 | 59 | 98 | 97 | 98 |
| 25° C./60% | 01 | 27 | 24 | 25 | 62 | 56 | 59 | 103* | 96 | 100 |
| Rel. Hum. | | | | | | | | | | |
| | 02 | 26 | 24 | 25 | 62 | 59 | 60 | 102* | 98 | 100 |
| | 03 | 25 | 23 | 24 | 58 | 55 | 57 | 96 | 91 | 94 |
| | 04 | 26 | 22 | 25 | 61 | 56 | 59 | 96 | 93 | 95 |
| 30° C./60% | 03 | NR | NR | NR | NR | NR | NR | NR | NR | NR |
| Rel. Hum. | | | | | | | | | | |
| 40° C./75% | 01 | 30 | 26 | 27 | 67 | 59 | 62 | 101* | 94 | 99 |
| Rel. Hum. | | | | | | | | | | |
| | 02 | 26 | 23 | 25 | 63 | 58 | 61 | 102* | 99 | 101* |
| | 03 | 26 | 23 | 25 | 64 | 55 | 59 | 85 | 69 | 76 |
| | 04 | 20 | 16 | 19 | 48 | 43 | 45 | 85 | 69 | 76 |

| | | | |
|---|---|---|---|
| Spec. Limits | Not More Than | 45%-75% | Not Less Than |
| | 40% | | 75% |

NR = Not required
(*percentage dissolution referenced to a baseline of 450 mg lithium carbonate per tablet. For tablet formulations exceeding this weight, calculated percentage greater than 100 percent is therefore possible.)

TABLE 19
Dissolution Stability Summary Lithium Carbonate - NaCMC -Glycine Formulations
Utilizing High Density Lithium in Current Commercial Packaging

| | | 1 Hour | | | 3 Hour | | | 7 Hour | | |
|---|---|---|---|---|---|---|---|---|---|---|
| Storage | Age, | Dissolution % | | | Dissolution % | | | Dissolution % | | |
| Conditions | (Months) | High | Low | Avg. | High | Low | Avg. | High | Low | Avg. |
| Initial | 00 | 31 | 28 | 29 | 66 | 62 | 64 | 102* | 99 | 100 |
| 25° C./60% | 01 | 28 | 23 | 26 | 65 | 55 | 61 | 103* | 94 | 99 |
| Rel. Hum. | | | | | | | | | | |
| | 02 | 26 | 25 | 26 | 64 | 61 | 62 | 103* | 98 | 101* |
| | 03 | 27 | 23 | 25 | 62 | 58 | 60 | 99 | 93 | 96 |
| | 04 | 26 | 24 | 25 | 62 | 59 | 60 | 99 | 96 | 97 |
| 30° C./60% | 03 | NR | NR | NR | NR | NR | NR | NR | NR | NR |
| Rel. Hum. | | | | | | | | | | |
| 40° C./75% | 01 | 28 | 25 | 26 | 68 | 62 | 65 | 106* | 102* | 104* |
| Rel. Hum. | | | | | | | | | | |
| | 02 | 29 | 25 | 26 | 67 | 63 | 65 | 104* | 102* | 103* |
| | 03 | 23 | 20 | 22 | 55 | 51 | 54 | 100 | 85 | 94 |
| | 04 | 22 | 19 | 20 | 53 | 49 | 51 | 96 | 88 | 93 |

| | | | |
|---|---|---|---|
| Spec. Limits | Not More Than | 45%-75% | Not Less Than |
| | 40% | | 75% |

NR = Not required
(*percentage dissolution referenced to a baseline of 450 mg lithium carbonate per tablet. For tablet formulations exceeding this weight, calculated percentage greater than 100 percent is therefore possible.)

### EXAMPLE V

### Lithium Carbonate—NaCMC—Bowl Charge Modification

A modification to the manufacturing method of the preceding examples was made in an additonal series of experiments with the NaCMC granulation. Standard commercial testing preparations equivalent to a bowl charge of 40,000 tablets at 450 mg lithium carbonate per tablet were utilized. Varying amounts of lithium carbonate, in an amount equal to either 5, 10, or 15 mg/tablet, were removed from the fluid bed bowl. The amount removed from the fluid bed bowl was then dissolved in water containing the desired amount of NaCMC and returned to the final formulation as part of the granulation solution spray. The effect of the bowl charge modification on the formulation dissolution rate varied from the baseline of approximately 50% dissolution at three hours (no lithium removed from granulation bowl and sprayed back onto granulation mixture with NaCMC), to an increase in dissolution rates, as shown in Table 20, that increased to as much as 75% at three hours with a 15 mg bowl modification (i.e., an amount of lithium equal to 15 mg/tablet removed from bowl charge, dissolved with water containing NaCMC, and then sprayed back onto the granulation mixture). In keeping with the experimental goal of achieving a modest increase in three hour dissolution rates, a bowl modification of 10 mg/tablet was chosen for further experimentation with varying lithium densities, as shown in Table 21.

TABLE 20
Effect of Bowl Charge Modification on Dissolution
Rates of NaCMC-Glycine Granulations

| | | | Stearic | | | |
|---|---|---|---|---|---|---|
| | NaCMC | Bowl Modification | Acid | Dissolution % | | |
| Formula | mg/tablet | mg/tablet | mg/tablet | 1 Hr. | 3 Hr. | 7 Hr. |
| 0106980 | 25 mg | 5 mg | 2.4 mg | 24 | 59 | 97 |
| 0106978 | 25 mg | 10 mg | 2.4 mg | 26 | 63 | 100 |
| 0107231 | 25 mg | 15 mg | 4.8 mg | 32 | 75 | 101* |

(*percentage dissolution referenced to a baseline of 450 mg lithium carbonate per tablet. For tablet formulations exceeding this weight, calculated percentage greater than 100 percent is therefore possible.)

TABLE 21
Effect of Different Densities of Lithium NaCMC using Glycine
Granulations With 10 mg/tablet Bowl Charge Modification

| | | Stearic | Lithium | | | |
|---|---|---|---|---|---|---|
| | NaCMC | Acid | Bulk | Dissolution % | | |
| Formula | mg/tablet | mg/tablet | Density | 1 Hr. | 3 Hr. | 7 Hr. |
| 0200582 | 25 mg | 4.8 mg | 0.49 | 33 | 68 | 101* |
| 0200579 | 25 mg | 4.8 mg | 0.51 | 27 | 61 | 97 |
| 0200576 | 25 mg | 4.8 mg | 0.525 | 35 | 73 | 102* |
| 0200755 | 25 mg | 4.8 mg | 0.55 | 35 | 70 | 103* |
| 0200774 | 25 mg | 4.8 mg | 0.55 | 27 | 62 | 98 |

(*percentage dissolution referenced to a baseline of 450 mg lithium carbonate per tablet. For tablet formulations exceeding this weight, calculated percentage greater than 100 percent is therefore possible.)

Dissolution stability studies were undertaken for both low density and high density lithium, utilizing the lithium carbonate—NaCMC—10 mg/tablet bowl charge modification formulations, as seen in Tables 22-24. The results of these studies, shown in Table 22-24, showed an improved dissolution rate in most time periods and storage conditions compared to the dissolution stabilities seen with the lithium carbonate—NaCMC formulations of Table 8. There was, however, a drop in dissolution rates at the highest experimental temperatures and relative humidity. This effect showed slight variation in two different granulation lots of high density lithium carbonate (Lot 0200755, manufactured in 2002; reported in Table 23 and Lot 0106978, manufactured in 2001).

TABLE 22
Stability Summary of Low Density Lithium Carbonate - NaCMC
Formulations With 10 mg/tablet Bowl Charge Modification

| | | 1 Hour | | | 3 Hour | | | 7 Hour | | |
|---|---|---|---|---|---|---|---|---|---|---|
| Storage | Age | Dissolution % | | | Dissolution % | | | Dissolution % | | |
| Conditions | (Months) | High | Low | Avg. | High | Low | Avg. | High | Low | Avg. |
| Initial | 00 | 40 | 32 | 37 | 74 | 67 | 71 | 103* | 100 | 102* |
| 25° C./60% | 01 | 34 | 28 | 32 | 70 | 66 | 68 | 103* | 100 | 101* |
| Rel. Hum. | | | | | | | | | | |
| | 02 | 36 | 29 | 33 | 71 | 64 | 68 | 103* | 81 | 97 |
| | 03 | 33 | 28 | 31 | 66 | 62 | 64 | 97 | 95 | 96 |
| | 04 | 36 | 25 | 32 | 71 | 65 | 68 | 97 | 91 | 93 |
| 30° C./60% | 03 | NR | NR | NR | NR | NR | NR | NR | NR | NR |
| Rel. Hum. | | | | | | | | | | |
| 40° C./75% | 01 | 35 | 33 | 31 | 72 | 67 | 69 | 102* | 99 | 100 |
| Rel. Hum. | | | | | | | | | | |
| | 02 | 40 | 30 | 33 | 74 | 64 | 67 | 104* | 97 | 102* |
| | 03 | 40 | 29 | 35 | 78 | 64 | 70 | 95 | 84 | 87 |
| | 04 | 25 | 24 | 25 | 55 | 51 | 53 | 95 | 84 | 87 |

| | | | |
|---|---|---|---|
| Spec. Limits | Not More Than | 45%-75% | Not Less Than |
| | 40% | | 75% |

NR = Not Required.
(*percentage dissolution referenced to a baseline of 450 mg lithium carbonate per tablet. For tablet formulations exceeding this weight, calculated percentage greater than 100 percent is therefore possible.)

TABLE 23
Stability Summary of High Density Lithium Carbonate - NaCMC Formulations
With 10 mg/tablet Bowl Charge Modification - Lot 0200755

| | | 1 Hour | | | 3 Hour | | | 7 Hour | | |
|---|---|---|---|---|---|---|---|---|---|---|
| Storage | Age | Dissolution % | | | Dissolution % | | | Dissolution % | | |
| Conditions | (Months) | High | Low | Avg. | High | Low | Avg. | High | Low | Avg. |
| Initial | 00 | 29 | 26 | 28 | 67 | 63 | 65 | 98 | 98 | 98 |
| 25° C./60% | 01 | 29 | 26 | 28 | 69 | 62 | 65 | 103* | 100 | 102* |
| Rel. Hum. | | | | | | | | | | |
| | 02 | 30 | 25 | 28 | 70 | 53 | 64 | 101* | 88 | 94 |
| | 03 | 29 | 26 | 27 | 66 | 63 | 65 | 99 | 92 | 97 |
| | 04 | 31 | 28 | 29 | 71 | 67 | 69 | 101* | 99 | 100 |
| 30° C./60% | 03 | NR | NR | NR | NR | NR | NR | NR | NR | NR |
| Rel. Hum. | | | | | | | | | | |
| 40° C./75% | 01 | 41 | 28 | 31 | 81 | 69 | 72 | 103* | 99 | 101* |
| Rel. Hum. | | | | | | | | | | |
| | 02. | 27 | 25 | 26 | 64 | 61 | 62 | 99 | 95 | 97 |
| | 03 | 25 | 21 | 23 | 54 | 50 | 52 | 95 | 87 | 90 |
| | 04 | 17 | 17 | 17 | 38 | 36 | 37 | 60 | 56 | 58 |

| | | | |
|---|---|---|---|
| Spec. Limits | Not More Than | 45%-75% | Not Less Than |
| | 40% | | 75% |

NR= Not required
(*percentage dissolution referenced to a baseline of 450 mg lithium carbonate per tablet. For tablet formulations exceeding this weight, calculated percentage greater than 100 percent is therefore possible.)

TABLE 24
Stability Summary of Appendix G High Density Lithium Carbonate - NaCMC
Formulations With 10 mg/tablet Bowl Charge Modification - Lot 0106978

| | | 1 Hour | | | 3 Hour | | | 7 Hour | | |
|---|---|---|---|---|---|---|---|---|---|---|
| Storage | Age | Dissolution % | | | Dissolution % | | | Dissolution % | | |
| Conditions | (Months) | High | Low | Avg. | High | Low | Avg. | High | Low | Avg. |
| Initial | 00 | 30 | 26 | 29 | 65 | 61 | 63 | 100 | 96 | 98 |
| 25° C./60% | 01 | 34 | 29 | 31 | 67 | 65 | 66 | 105* | 100 | 103* |
| Rel. Hum. | | | | | | | | | | |
| | 02 | 31 | 29 | 30 | 68 | 66 | 67 | 107* | 101* | 103* |
| | 03 | 30 | 29 | 30 | 64 | 63 | 64 | 100 | 95 | 98 |
| | 04 | 29 | 27 | 28 | 66 | 62 | 64 | 101* | 99 | 100 |
| 30° C./60% | 03 | NR | NR | NR | NR | NR | NR | NR | NR | NR |
| Rel. Hum. | | | | | | | | | | |
| 40° C./75% | 01 | 30 | 29 | 30 | 67 | 64 | 65 | 101* | 96 | 99 |
| Rel. Hum. | | | | | | | | | | |
| | 02 | 30 | 27. | 28 | 65 | 55 | 62 | 101* | 96 | 99 |
| | 03 | 24 | 22 | 23 | 52 | 49 | 51 | 92 | 85 | 88 |
| | 04 | 21 | 20 | 20 | 46 | 43 | 45 | 84 | 74 | 79 |

| | | | |
|---|---|---|---|
| Spec. Limits | Not More Than | 45%-75% | Not Less Than |
| | 40% | | 75% |

NR = Not required
(*percentage dissolution referenced to a baseline of 450 mg lithium carbonate per tablet. For tablet formulations exceeding this weight, calculated percentage greater than 100 percent is therefore possible.)

### EXAMPLE VI

### Dissolution Profiles With Altered Commercial Packaging

The effects of product packaging was hypothesized to play a role in the changes in dissolution profiles seen over time with various lithium carbonate formulations. In the first experiment, as a control, Eskalith CR 450® from several lots, in the current commercial packaging of Eskalith CR®, was placed on stability. All sample performed outside of specifications after six months testing at high levels of heat and humidity, with representative studies shown in Table 25.

TABLE 25
Stability Summary of Currently Marketed, Gelatinized Lithium Carbonate
(Eskalith CR 450 ®) in Current Commercial Packaging

| | | | 1 Hour | | | 3 Hour | | | 7 Hour | | |
|---|---|---|---|---|---|---|---|---|---|---|---|
| Storage | Age | | Dissolution % | | | Dissolution % | | | Dissolution % | | |
| Conditions | (Months) | Potency | High | Low | Avg. | High | Low | Avg. | High | Low | Avg. |
| Initial | 00 | 98.3 | 26 | 22 | 24 | 70 | 57 | 64 | 99 | 96 | 97 |
| 25° C./60% | 01 | NR | 24 | 20 | 22 | 65 | 59 | 63 | 100 | 95 | 98 |
| Rel. Hum. | | | | | | | | | | | |
| | 02 | NR | 27 | 23 | 25 | 65 | 60 | 63 | 102* | 98 | 101* |
| | 03 | 97.3 | 23 | 21 | 22 | 60 | 54 | 58 | 100 | 94 | 97 |
| | 06 | 97.5 | 22 | 20 | 21 | 58 | 52 | 54 | 96 | 91 | 95 |
| | 09 | 98.6 | 22 | 20 | 21 | 57 | 54 | 55 | 96 | 92 | 94 |
| | 12 | 99.7 | 20 | 19 | 20 | 57 | 52 | 54 | 98 | 93 | 95 |
| 30° C./60% | 03 | NR | NR | NR | NR | NR | NR | NR | NR | NR | NR |
| Rel. Hum. | | | | | | | | | | | |
| | 06 | 97.4 | 20 | 18 | 19 | 52 | 46 | 49 | 99 | 94 | 96 |
| | 09 | 99.9 | 21 | 18 | 20 | 55 | 53 | 54 | 97 | 92 | 95 |
| | 12 | 100.0 | 19 | 17 | 18 | 55 | 49 | 51 | 92 | 88 | 90 |
| 40° C./75% | 03 | 99.3 | 20 | 19 | 19 | 50 | 46 | 48 | 89 | 84 | 87 |
| Rel. Hum. | | | | | | | | | | | |
| | 06 | 97.8 | 18 | 16 | 17 | 40 | 38 | 39 | 79 | 73 | 76 |

| | | | | |
|---|---|---|---|---|
| Spec. Limits | 99.0-110.0 | Not More Than | 45%-75% | Not Less Than |
| | | 40% | | 75% |

NR = Not required
(*percentage dissolution referenced to a baseline of 450 mg lithium carbonate per tablet. For tablet formulations exceeding this weight, calculated percentage greater than 100 percent are therefore possible.)

In accompanying experiments, the effects of altering the current commercial packaging of the currently available, gelatinized, form of lithium carbonate (Eskalith CR 450®) was studied in an attempt to develop improved dissolution profiles, particularly at longer storage times at highter heat and humidity levels, than those seen in the representative baseline reported in Table 25. In this follow up study, the currently marketed packaging of Eskalith CR 450®, consisting of a 100 cc, High Density Polyethylene (HDPE) white bottle with a 33 mm white polypropylene plastic cap, desiccant, and cotton fill was modified with the addition of an induction heat seal and two, 2 in 1 desiccant canisters placed within the bottle. This modification of the current commercial packaging, carried out in three separate experiments, as shown in Tables 26-28, showed considerable improvement in the three hour dissolution profiles of the currently marketed gelatinized form of lithium carbonate studied (Eskalith CR 450®), whose baseline values are shown in Table 25. In the test, averages in the modified packaging performed within specifications after six months storage at high temperature and humidity.

TABLE 26
Stability Summary of Currently Marketed, Gelatinized Lithium Carbonate (Eskalith
CR 450 ®) in Proposed Commercial Packaging (Addition of Induction
Heat Seal and Two, 2 in 1 Desiccant Canisters) - Lot 0102583

| | | | 1 Hour | | | 3 Hour | | | 7 Hour | | |
|---|---|---|---|---|---|---|---|---|---|---|---|
| Storage | Age | | Dissolution % | | | Dissolution % | | | Dissolution % | | |
| Conditions | (Months) | Potency | High | Low | Avg. | High | Low | Avg. | High | Low | Avg. |
| Initial | 00 | 102.8 | 27 | 24 | 26 | 69 | 65 | 67 | 105* | 101* | 103* |
| 25° C./60% | 03 | 103.3 | 28 | 23 | 25 | 73 | 61 | 67 | 112* | 102* | 106* |
| Rel. Hum. | | | | | | | | | | | |
| | 06 | 102.3 | 28 | 24 | 26 | 73 | 61 | 67 | 112* | 102* | 106* |
| 30° C./60% | 01 | 99.9 | 27 | 25 | 26 | 73 | 67 | 69 | 103* | 99 | 101* |
| Rel. Hum. | | | | | | | | | | | |
| | 02 | 102.3 | 28 | 26 | 27 | 72 | 65 | 69 | 104* | 102* | 103* |
| | 03 | 101.4 | 34 | 26 | 29 | 75 | 70 | 73 | 104* | 100 | 102* |
| | 06 | 102.5 | 25 | 24 | 24 | 67 | 64 | 65 | 105* | 102* | 103* |
| 40° C./75% | 01 | 101.2 | 26 | 23 | 24 | 68 | 61 | 64 | 105* | 102* | 103* |
| Rel. Hum. | | | | | | | | | | | |
| | 02 | 101.4 | 23 | 22 | 23 | 62 | 58 | 60 | 102* | 100 | 101* |
| | 03 | 100.9 | 22 | 20 | 21 | 59 | 55 | 57 | 102* | 98 | 100 |
| | 06 | 102.9 | 20 | 18 | 19 | 55 | 52 | 54 | 100 | 96 | 98 |

| | | | | |
|---|---|---|---|---|
| Spec. Limits | 99.0-110.0 | Not More Than | 45%-75% | Not Less Than |
| | | 40% | | 75% |

(*percentage dissolution referenced to a baseline of 450 mg lithium carbonate per tablet. For tablet formulations exceeding this weight, calculated percentage greater than 100 percent is therefore possible.)

TABLE 27
Stability Summary of Currently Marketed, Gelatinized Lithium Carbonate (Eskalith
CR 450 ®) in Proposed Commercial Packaging (Addition of Induction
Heat Seal and Two, 2 in 1 Desiccant Canisters) - Lot 0102584

| | | | 1 Hour | | | 3 Hour | | | 7 Hour | | |
|---|---|---|---|---|---|---|---|---|---|---|---|
| Storage | Age | | Dissolution % | | | Dissolution % | | | Dissolution % | | |
| Conditions | (Months) | Potency | High | Low | Avg. | High | Low | Avg. | High | Low | Avg. |
| Initial | 00 | 103.7 | 25 | 23 | 24 | 64 | 60 | 63 | 104* | 97 | 102* |
| 25° C./60% | 03 | 103 | 27 | 23 | 25 | 70 | 63 | 66 | 106* | 98 | 104* |
| Rel. Hum. | | | | | | | | | | | |
| | 06 | 102.5 | 25 | 23 | 24 | 67 | 57 | 63 | 106* | 98 | 104* |
| 30° C./60% | 01 | 99.9 | 26 | 24 | 25 | 66 | 62 | 64 | 102* | 99 | 101* |
| Rel. Hum. | | | | | | | | | | | |
| | 02 | 101.5 | 27 | 24 | 25 | 67 | 60 | 63 | 103* | 100 | 101* |
| | 03 | 101.8 | 25 | 22 | 23 | 65 | 59 | 62 | 102* | 100 | 101* |
| | 06 | 101.9 | 24 | 22 | 23 | 67 | 63 | 65 | 106* | 102* | 104* |
| 40° C./75% | 01 | 101 | 25 | 22 | 24 | 64 | 61 | 62 | 100 | 98 | 99 |
| Rel. Hum. | | | | | | | | | | | |
| | 02 | 101.2 | 23 | 21 | 22 | 59 | 53 | 56 | 100 | 95 | 97 |
| | 03 | 100.3 | 29 | 22 | 24 | 71 | 55 | 61 | 103* | 94 | 100 |
| | 06 | 101.7 | 19 | 16 | 18 | 51 | 43 | 48 | 97 | 83 | 91 |

| | | | | |
|---|---|---|---|---|
| Spec. Limits | 99.0-110.0 | Not More Than | 45%-75% | Not Less Than |
| | | 40% | | 75% |

(*percentage dissolution referenced to a baseline of 450 mg lithium carbonate per tablet. For tablet formulations exceeding this weight, calculated percentage greater than 100 percent is therefore possible.)

TABLE 28
Stability Summary of Currently Marketed, Gelatinized Lithium Carbonate (Eskalith
CR 450 ®) in Proposed Commercial Packaging (Addition of Induction
Heat Seal and Two, 2 in 1 Desiccant Canisters) - Lot 0102588

| | | | 1 Hour | | | 3 Hour | | | 7 Hour | | |
|---|---|---|---|---|---|---|---|---|---|---|---|
| Storage | Age | | Dissolution % | | | Dissolution % | | | Dissolution % | | |
| Conditions | (Months) | Potency | High | Low | Avg. | High | Low | Avg. | High | Low | Avg. |
| Initial | 00 | 102.8 | 26 | 21 | 24 | 68 | 55 | 63 | 102* | 98 | 101* |
| 25° C./60% | 03 | 103.2 | 29 | 23 | 26 | 75 | 62 | 68 | 102* | 98 | 100 |
| Rel. Hum. | | | | | | | | | | | |
| | 06 | 102.6 | 27 | 21 | 24 | 68 | 58 | 64 | 109* | 97 | 103* |
| 30° C./60% | 01 | 99.4 | 26 | 24 | 25 | 66 | 60 | 63 | 101* | 99 | 100 |
| Rel. Hum. | | | | | | | | | | | |
| | 02 | 101.2 | 24 | 22 | 23 | 64 | 63 | 64 | 103* | 101* | 102* |
| | 03 | 101.3 | 25 | 22 | 23 | 66 | 61 | 63 | 101* | 98 | 99 |
| | 06 | 102.1 | 28 | 22 | 25 | 76 | 61 | 68 | 107* | 99 | 103* |
| 40° C./75% | 01 | 100.7 | 24 | 22 | 23 | 67 | 59 | 62 | 100 | 97 | 99 |
| Rel. Hum. | | | | | | | | | | | |
| | 02 | 101.4 | 23 | 22 | 22 | 62 | 60 | 61 | 102* | 99 | 100 |
| | 03 | 101.5 | 22 | 18 | 21 | 65 | 61 | 63 | 104* | 100 | 102* |
| | 06 | 101.9 | 18 | 16 | 17 | 55 | 48 | 52 | 102* | 95 | 97 |

| | | | | |
|---|---|---|---|---|
| Spec. Limits | 99.0-110.0 | Not More Than | 45%-75% | Not Less Than |
| | | 40% | | 75% |

(*percentage dissolution referenced to a baseline or 450 mg lithium carbonate per tablet. For tablet formulations exceeding this weight, calculated percentage greater than 100 percent is therefore possible.)

### INDUSTRIAL APPLICABILITY

While many drugs are conveniently dosed using conventional delayed release or sustained release technology, pharmaceuticals such as lithium compounds that have highly variable dissolution rates and narrow ranges of clinically therapeutic plasma concentrations present difficult problems. The present inventors, have, through an extensive amount of research, determined that a formulation of lithium carbonate including the excipients Sodium Carboxymethylcellulose (NaCMC) and glycine has an enhanced dissolution profile at three hours, compared to a formulation not containing glycine, and that the formulation including glycine has a more stable three hour dissolution profile following prolonged periods of storage under varying conditions. In addition, a process modification wherein approximately 10 mg/tablet of the lithium—NaCMC granulation is removed from the fluid bed, solubilized, and then top sprayed on the remaining granulation, also exhibits an improved three hour dissolution profile, and that the formulation produced by this method has a more stable three hour dissolution profile following prolonged periods of storage under varying conditions. Testing of the dissolution stability of a currently marketed lithium carbonate formulation in a modified packaging indicates that the improved dissolution stability profile of lithium carbonate—NaCMC, lithium carbonate NaCMC—glycine, and lithium carbonate—NaCMC formulated in a bowl charge modification process may all be further improved with modification of the current commercial packaging of lithium carbonate formulations.

The compositions of the present invention may be administered according to various dosage regiments, i.e., once-daily or multiple daily occurrences (e.g., two), or at various intervals (e.g., every 12 hours). The amount of lithium carbonate employed per dosage form (e.g., tablet) may vary and include, without limitation, 300 mg and 450 mg. The compositions may be employed in various dosage forms including, without limitation, tablets, pills, powders, elixirs, suspensions, emulsions, solutions, syrups, capsules (such as, for example, soft and hard gelatin capsules), suppositories, sterile injectable solutions, and sterile packaged powders.

Having thus described the present invention in detail, it will be obvious to those skilled in the art that various changes or modifications may be made without departing from the scope of the invention define in the appended claims and described in the specification.

We claim:

1. A pharmaceutical composition for oral administration, comprising:
a. lithium carbonate,
b. optional pharmacologic excipients,
c. at least one dissolution rate stabilizer, and
d. at least one secondary release agent.

2. The pharmaceutical composition according to claim 1, wherein the lithium carbonate does not exceed a dose greater than about 450 mg/tablet.

3. The pharmacologic composition according to claim 1 additionally comprising iron oxide as a colorant, wherein the iron oxide does not exceed a level of about 1 mg/tablet.

4. The pharmaceutical composition according to claim 1, wherein the optional pharmacologic excipients further comprises at least one lubricant at a concentration of between about 0.1% and about 1.0% of the composition by weight.

5. The pharmaceutical composition according to claim 4 wherein said lubricant is selected from stearic acid, calcium stearate, magnesium stearate and sodium stearyl fumerate, said lubricant is at a concentration of about 0.1% to about 1.0% of the composition by weight.

6. The pharmaceutical composition according to claim 1, wherein the composition is compressed at a pressure of between about 7 kPa to about 20 kPa.

7. The pharmaceutical composition according to claim 1, wherein the composition is compressed with a pressure not greater than about 7 kPa.

8. The pharmaceutical composition according to claim 1, wherein the at least one dissolution rate stabilizer comprises sodium carboxymethylcellulose.

9. The pharmaceutical composition according to claim 8, wherein the sodium carboxymethylcellulose comprises between about 5% to about 15% of the composition by weight.

10. The pharmaceutical composition according to claim 8, wherein the sodium carboxymethylcellulose comprises not more than about 5% of the composition by weight.

11. The pharmaceutical composition according to claim 1, wherein the at least one secondary release agent comprises glycine.

12. The pharmaceutical composition according to claim 11, wherein the glycine comprises between about 0.5 to about 40 mg/tablet.

13. The pharmaceutical composition according to claim 11, wherein the glycine comprises a level not greater than about 20 mg/tablet.

14. The pharmaceutical composition according to claim 11, wherein the glycine comprises a level not greater than about 14 mg/tablet.

15. The pharmaceutical composition according to claim 11, wherein the glycine comprises a level not greater than about 11 mg/tablet.

16. The pharmaceutical composition according to claim 11, wherein the glycine comprises a level not greater than about 2 mg/tablet.

17. A pharmaceutical composition for oral administration, comprising:
a. lithium carbonate,
b. iron oxide,
c. stearic acid,
d. sodium carboxymethylcellulose,
e. glycine and
f. optional pharmaceutically acceptable excipients.

18. A controlled release solid dosage form of lithium carbonate containing:
a. about 85% to about 90% by weight lithium carbonate,
b. about 10% to about 15% sodium carboxymethylcellulose,
c. about 0.5% glycine, and
d. optional pharmaceutically acceptable excipients.

19. A process for preparing a controlled release solid dosage form of lithium carbonate in accordance to claim 1, which comprises the steps of:
a. mixing lithium carbonate and iron oxide into a blend;
b. solubilizing a solution of water, sodium carboxymethylcellulose, and at least one secondary release agent;
c. placing the blend of lithium carbonate and iron oxide in a bed of a fluid bed granulator;
d. creating a top sprayed blend by spraying the solution onto the blend in the bed of the fluid bed granulator;
e. granulating the top sprayed blend in the fluid bed granulator into a granulation;
f. forming a composition by milling the granulation with at least one excipient; and
g. pressing the granulation with at least one excipient into tablets in a tablet press.

20. The process according to claim 19, wherein the at least one excipient comprises at least one lubricant.

21. The process according to claim 20, wherein the at least one lubricant is selected from the group consisting of stearic acid, sodium stearyl fumerate, calcium stearate, and magnesium stearate.

22. The process according to claim 21, wherein the secondary release agent comprises glycine.

23. A pharmaceutical composition for oral administration prepared according to claim 22.

24. A method for the treatment of Bipolar Disorder consisting essentially of orally administering to a patient a therapeutically effective amount of a composition according to claim 1.

25. A method for the treatment of Manic-Depressive Disorder consisting essentially of orally administering to a patient a therapeutically effective amount of a composition according to claim 23.

26. A method for the treatment of Bipolar Disorder consisting essentially of orally administering to a patient a therapeutically effective amount of a controlled release solid dosage form of lithium carbonate according to claim 8.

27. A process for manufacturing a pharmaceutical composition for oral administration, which comprises the steps of:
a. mixing lithium carbonate and iron oxide into a first blend,
b. solubilizing a first solution of water and sodium carboxymethylcellulose,
c. reserving a quantity of the first blend equal to create a second blend,
d. placing the first blend of lithium carbonate in a bed of a fluid bed granulator,
e. dissolving the second blend in the first solution of water and sodium carboxymethylcellulose to form a second solution,
f. creating a top sprayed blend by top spraying the second solution onto the first blend in the bed of the fluid bed granulator,
g. granulating the top sprayed blend in the fluid bed granulator into a granulation,
h. milling the granulation with at least one excipient, and
i. pressing the granulation with at least one excipient composition into tablets in a tablet press.

28. The process according to claim 27 wherein the at least one excipient comprises at least one lubricant.

29. The process according to claim 28, wherein the at least one lubricant is selected from the group consisting of stearic acid, sodium stearyl fumerate, calcium stearate, and magnesium stearate.

30. A pharmaceutical composition for oral administration prepared according to claim 27.

31. A pharmaceutical composition for oral administration prepared according to claim 28.

32. A pharmaceutical composition for oral administration prepared according to claim 29.

33. A method for the treatment of Bipolar Disorder consisting essentially of orally administering to a patient a therapeutically effective amount of a composition according to claim 30.

34. A method for the treatment of Manic-Depressive Disorder consisting essentially of orally administering to a patient a therapeutically effective amount of a composition according to claim 31.

35. A method for the treatment of Bipolar Disorder consisting essentially of orally administering to a patient a therapeutically effective amount of a composition according to claim 32.

36. A pharmaceutical composition for oral administration, comprising:
a. lithium carbonate,
b. optional pharmacological excipients, and
c. at least one dissolution rate stabilizer.

37. The pharmaceutical composition according to claim 36, wherein the lithium carbonate does not exceed a dose greater than about 450 mg/tablet.

38. The pharmacologic composition according to claim 36, additionally comprising iron oxide as a colorant, wherein the iron oxide does not exceed a level of about 1 mg/tablet.

39. The pharmacologic composition of claim 36, wherein the optional pharmacologic excipients further comprises at least one lubricant.

40. The pharmaceutical composition according to claim 39, wherein the at least one lubricant further comprises stearic acid at 0.1% to about 5.0% of the composition by weight.

41. The pharmaceutical composition according to claim 40, wherein the at least one lubricant further comprises stearic acid at a level consisting of not more than about 0.5% of the composition by weight.

42. The pharmaceutical composition according to claim 39, wherein the at least one lubricant further comprises sodium stearyl fumerate.

43. The pharmaceutical composition according to claim 42, wherein the at least one lubricant further comprises sodium stearyl fumerate at a level of about 0.1% to about 1.0% of the composition by weight.

44. The pharmaceutical composition according to claim 43, wherein the at least one lubricant further comprises sodium stearyl fumerate at a level consisting of not more than about 0.5% of the composition by weight.

45. The pharmaceutical composition according to claim 39, wherein the at least one lubricant further comprises calcium stearate at a level of about 0.1% to about 1.0% of the composition by weight.

46. The pharmaceutical composition according to claim 39, wherein the at least one lubricant further comprises magnesium stearate at a level of about 0.1% to about 1.0% of the composition by weight.

47. The pharmaceutical composition according to claim 36, wherein the composition is compressed at a hardness of between about 7 kPa to about 20 kPa.

48. The pharmaceutical composition according to claim 36, wherein the composition is compressed at a pressure not greater than about 7 kPa.

49. The pharmacologic composition according to claim 36, wherein the at least one dissolution rate stabilizer comprises sodium carboxymethylcellulose.

50. The pharmaceutical composition according to claim 49, wherein the sodium carboxymethylcellulose comprises between about 5% to about 15% of the composition by weight.

51. The pharmaceutical composition according to claim 49, wherein the sodium carboxymethylcellulose comprises not more than about 10% of the composition by weight.

52. The pharmaceutical composition according to claim 49, wherein the sodium carboxymethylcellulose comprises not more than about 5% of the composition by weight.

53. A pharmaceutical composition for oral administration, comprising:
a. about 85% to about 90% by weight lithium carbonate,
b. about 10% to about 15% sodium carboxymethylcellulose,
c. iron oxide,
d. stearic acid, and
e. optional pharmaceutically acceptable excipients.

54. A process for manufacturing a pharmaceutical composition for oral administration, in accordance with claim 53, which comprises the steps of.
a. mixing lithium carbonate and iron oxide into a blend,
b. solubilizing a solution of water and sodium carboxymethylcellulose,
c. placing the blend of lithium carbonate and iron oxide in a bed of a fluid bed granulator,
d. creating a granulation by top spraying the solution onto the blend in the bed of the fluid bed granulator,
e. forming a composition by milling the granulation with at lest one excipient, and
f. pressing the granulation with at least one excipient composition into tablets in a tablet press.

55. The process according to claim 54 wherein the at least one excipient comprises at least one lubricant.

56. The process of claim 54, wherein the at least one excipient is a lubricant selected from the group consisting of stearic acid, sodium stearyl fumerate, calcium stearate, and magnesium stearate.

57. A pharmaceutical composition for oral administration prepared according to claim 54.

58. A pharmaceutical composition for oral administration prepared according to claim 55.

59. A method for the treatment of Bipolar Disorder (Manic-Depressive Disorder) consisting essentially of orally administering to a patient a therapeutically effective amount of a composition according to claim 36.

60. A method for the treatment of Bipolar Disorder (Manic-Depressive Disorder) consisting essentially of orally administering to a patient a therapeutically effective amount of a composition according to claim 53.
